(19) United States
(12) Patent Application Publication
(10) Pub. No.: US 2025/0239343 A1
(43) Pub. Date: Jul. 24, 2025

(54) SYSTEM AND METHOD FOR WOUND TRIAGING AND RECOMMENDATIONS FOR TREATMENTS

(71) Applicant: Adiuvo Diagnostics Private Limited, Chennai (IN)

(72) Inventors: Bala Pesala, Chennai (IN); Geethanjali Radhakrishnan, Chennai (IN)

(21) Appl. No.: 18/854,082

(22) PCT Filed: Apr. 4, 2023
(86) PCT No.: PCT/IN2023/050328

§ 371 Date: Oct. 4, 2024

(30) Foreign Application Priority Data
Apr. 4, 2022 (IN) 202241006099
Apr. 4, 2023 (WO) PCT/IN2023/050328

(51) Int. Cl. G16H 20/00; A61B 5/00; G06T 7/00; G06V 10/764; G06V 30/148; G16H 10/20; G16H 10/60; G16H 15/00; G16H 50/30

(52) U.S. Cl. CPC G16H 20/00; A61B 5/445; A61B 5/4842; A61B 5/4848; A61B 5/7264; A61B 5/7275; G06T 7/0014; G06V 10/764; G06V 30/148; G16H 10/20; G16H 10/60; G16H 15/00; G16H 50/30; G06T 2207/20081; G06T 2207/30088; G06V 2201/03

(57) ABSTRACT

A wound triaging and recommendation system for wound triaging and recommendation for treatments is disclosed. A wound triaging and recommendation system includes a text and voice based conversational artificial intelligence (AI) subsystem determines data associated with at least one of: genetic predisposition, the one or more diseases influencing a wound, and a state of the wound. A wound image analytics subsystem that determines severity and risk category of wound using a machine learning algorithm. An AI based text and image analytics subsystem extracts key clinical parameters and changes in key clinical parameters over time using optical character recognition techniques. A patient treatment recommendation subsystem triages the wound to identify the severity of wound by qualifying risk score for the wound, to provide wound prognostics for wound healing, and to provide treatment recommendations associated with personalized therapeutic routes for wound healing to the patient.

### EARLIEST PRIORITY DATE

This Application claims priority from a patent application filed in India having application Ser. No. 20/224,1006099, on Apr. 4, 2022, and titled “WOUND TRIAGING AND RECOMMENDATION SYSTEM AND A METHOD THEREOF” and PCT Application bearing no. “PCT/IN2023/050328” filed on Apr. 4, 2023, titled “WOUND TRIAGING AND RECOMMENDATION SYSTEM AND A METHOD THEREOF”

### FIELD OF INVENTION

Embodiments of the present disclosure relates to healthcare systems and more particularly relates to wound triaging and recommendation system and a method thereof.

### BACKGROUND

An escalating physiological, psychological, social, and financial burdens of wounds and wound care on patients, families, and society demands attention in healthcare sector. Many forces affect changes in healthcare provision for the patients with chronic wounds including managed care, limited number of wound care therapists, increasingly ageing and disabled population, regulatory and malpractice issues, and compromised care. Additionally, the wounds such as diabetic foot ulcers, surgical site infections, burns and the like, require special care such as continuous monitoring looking for all potential infections and tailoring treatments to ensure the patient heals fast. Especially, patients with diabetes represent a precarious population of >500 million worldwide. Diabetes patients have a higher risk of morbidity and mortality in general and especially from emerging infectious diseases such as COVID-19.

Therefore, decreasing hospital visits of the patients by differentiating those with life or limb threatening (infectious diseases society of America (IDSA) grade 3 and 4) infections from non-limb threatening infections forms the basis of wound triaging. Wound care centres away from the hospitals can take care of most patients expect for the patients in critical state. However, the patients in the critical state require in-patients visit to an expert doctor clinic which takes a lot of time, effort, and cost to diagnose the wound of the patient. Additionally, the doctors must consider multiple factors such as grade of the wound of the patient, infection level of the wound of the patient, medicines take by the patient for the wound, other co-morbidities, and the like for tailoring the treatment options.

Therefore, there is a need for an improved wound triaging and recommendation system and a method thereof to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with one embodiment of the disclosure, a wound triaging and recommendation system for wound triaging and recommendation for treatments is disclosed. The wound triaging and recommendation system includes a hardware processor, and a memory that is coupled to the hardware processor. The memory includes a set of program instructions in the form of a plurality of subsystems, configured to be executed by the hardware processor. The plurality of subsystems include a text and voice based conversational artificial intelligence (AI) subsystem, a wound image analytics subsystem, an AI based text and image analytics subsystem, and a patient treatment recommendation subsystem.

The text and voice based conversational AI subsystem obtain patient's medical data including at least one of: history of one or more diseases of a patient, family information of the patient, symptoms of the one or more diseases in the patient, and medicines consumed by the patient through a user device of the patient. The text and voice based conversational AI subsystem determines data associated with at least one of: genetic predisposition, the one or more diseases influencing a wound, and a state of the wound based on an effect of a current medicine that the patient consumes based on the obtained patient's medical data using a machine learning algorithm.

The wound image analytics subsystem collects images of the wound from the patient. The wound image analytics subsystem classifies the wound of the patient into a plurality of categories including at least one of: granulation, necrotic, and cellulitis based on the collected images of the wound from the patient using the machine learning algorithm. The wound image analytics subsystem determines severity and risk category of the wound based on the classification of the wound of the patient using the machine learning algorithm.

The AI based text and image analytics subsystem obtains information associated with patient's clinical reports from the patient. The AI based text and image analytics subsystem extract key clinical parameters and changes in the key clinical parameters over time from the patient's clinical reports by scanning the patient's clinical reports using optical character recognition techniques.

The patient treatment recommendation subsystem obtains at least one of: (a) the determined data associated with at least one of: the genetic predisposition, the one or more diseases influencing the wound, and the state of the wound based on the effect of the current medicine that the patient consumes, (b) the determined severity and risk category of the wound, and (c) the extracted key clinical parameters and the changes in the key clinical parameters over time from at least one of: the text and voice based AI subsystem, the wound image analytics subsystem, and the AI based text and image analytics subsystem.

The patient treatment recommendation subsystem obtains medical data and reports from other patients. In an embodiment, the medical data and reports of the other patients include past medical history of the other patients. The patient treatment recommendation subsystem triages the wound to at least one of: identify the severity of the wound by qualifying risk score for the wound, provide wound prognostics for wound healing, and provide treatment recommendations associated with personalized therapeutic routes for healing the wound to the patient based on results outputted from at least one of: the text and voice based AI subsystem, the wound image analytics subsystem, the AI based text and image analytics subsystem, and the medical data and reports from other patients.

In one aspect, a wound triaging and recommendation method for wound triaging and recommendation for treatments using a wound triaging and recommendation system is disclosed. The wound triaging and recommendation method includes the following steps of: (a) determining, by the hardware processor, data associated with at least one of: genetic predisposition, the one or more diseases influencing a wound, and a state of the wound based on an effect of a current medicine that the patient consumes based on the obtained patient's medical data using a machine learning algorithm; (b) collecting, by the hardware processor, images of the wound from the patient; (c) classifying, by the hardware processor, the wound of the patient into a plurality of categories including at least one of: granulation, necrotic, and cellulitis based on the collected images of the wound from the patient using the machine learning algorithm; (d) determining, by the hardware processor, severity and risk category of the wound based on the classification of the wound of the patient using the machine learning algorithm; (e) obtaining, by the hardware processor, information associated with patient's clinical reports from the patient; (f) extracting, by the hardware processor, key clinical parameters and changes in the key clinical parameters over time from the patient's clinical reports by scanning the patient's clinical reports using optical character recognition techniques; (g) obtaining, by the hardware processor, at least one of: (i) determined data associated with at least one of: the genetic predisposition, the one or more diseases influencing the wound, and the state of the wound based on the effect of the current medicine that the patient consumes, (ii) the determined severity and risk category of the wound, and (iii) the extracted key clinical parameters and the changes in the key clinical parameters over time from at least one of: the text and voice based AI subsystem, the wound image analytics subsystem, and the AI based text and image analytics subsystem; (h) obtaining, by the hardware processor, medical data and reports from other patients, wherein the medical data and reports of other patients include past medical history of the other patients; and (i) triaging, by the hardware processor, the wound to at least one of: identify the severity of the wound by qualifying risk score for the wound, provide wound prognostics for wound healing, and provide treatment recommendations associated with personalized therapeutic routes for healing the wound to the patient based on results outputted from at least one of: the text and voice based AI subsystem, the wound image analytics subsystem, the AI based text and image analytics subsystem, and the medical data and reports from other patients.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a system architecture of a wound triaging and recommendation system for wound triaging and recommendations for treatments, in accordance with an embodiment of the present disclosure;

FIG. 2 is an exploded view of the wound triaging and recommendation system, such as those shown in FIG. 1, in accordance with an embodiment of the present disclosure;

FIG. 3 is a block diagram of the wound triaging and recommendation system, such as those shown in FIG. 1, in accordance with an embodiment of the present disclosure;

FIG. 4 is a schematic representation of an exemplary process flow depicting wound image analytics process, in accordance with an embodiment of the present disclosure;

FIG. 5 is a process flowchart illustrating an exemplary method of performing an artificial intelligence (AI) based image analytics, in accordance with an embodiment of the present disclosure;

FIG. 6 is a process flowchart illustrating an exemplary method of performing wound triaging and recommendation for treatments in case of digital assist or remote teleconsultation in a multi-user environment, in accordance with an embodiment of the present disclosure;

FIGS. 7A-7G are exemplary graphical user interfaces illustrating the wound triaging and recommendation system running on a user device, in accordance with an embodiment of the present disclosure;

FIG. 8 is a block diagram of a patient treatment recommendation subsystem based on the inputs collated from patients' data, wound image data, clinical reports and other patients data, in accordance with an embodiment of the present disclosure;

FIG. 9 is an exemplary view of the wound triaging and recommendation system (i.e., a wound digital assistant) to provide outputs of at least one of: a wound chronicity score, wound interventional strategies, and customized therapeutic strategies, in accordance with an embodiment of the present disclosure;

FIG. 10 is an exemplary view of the wound triaging and recommendation system to predict wound healing probability and healing time, in accordance with an embodiment of the present disclosure;

FIG. 11 is an exemplary view of the wound triaging and recommendation system to predict a type of the wound, and disease co-morbidity, and to provide a wound management education, in accordance with an embodiment of the present disclosure; and

FIG. 12 is a flow chart illustrating a computer implemented wound triaging and recommendation method for wound triaging and recommendation for treatments using a wound triaging and recommendation system, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

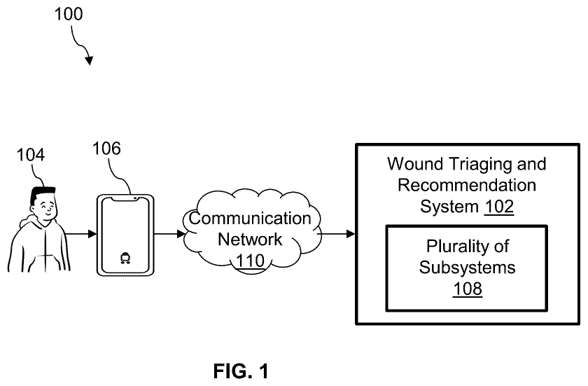

FIG. 1 is a system architecture 100 of a wound triaging and recommendation system 102 for wound triaging and recommendations for treatments, in accordance with an embodiment of the present disclosure. The system 100 includes a user 104, a user device 106, the wound triaging and recommendation system 102, and a communication network 110. The wound triaging and recommendation system 102 may include a plurality of subsystems 108 that help to triage the wound and recommend for treatments with the help of artificial intelligence (AI) models (e.g., a machine learning algorithm and a deep learning algorithm). In an embodiment, the wound triaging and recommendation system 102 may be installed in the user device 106. The user device 106 may be used by the user 104. In an embodiment, the user 104 may be at least one of: a patient, a physician, a doctor, and the like. In an embodiment, the user device 106 may be at least one of: a mobile phone, a personal computer (PC), a Smartphone, an electronic notebook, and the like. The system 100 further includes an explainable AI framework that is integrated in the system 100 for easy understanding of AI decisions by at least one of: the doctors, the physicians, and the patient, and the like. In one embodiment, the user 104 receives the recommendations for treatments from the wound triaging and recommendation system 102 through the communication network 110.

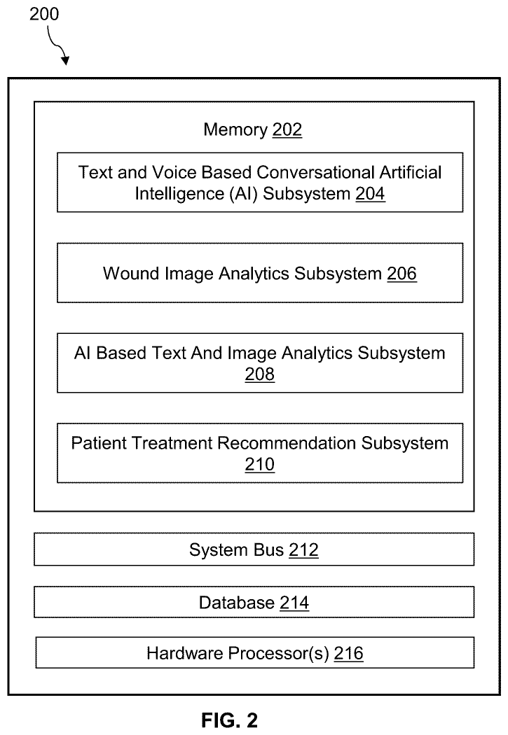

FIG. 2 is an exploded view 200 of the wound triaging and recommendation system 102, such as those shown in FIG. 1, in accordance with an embodiment of the present disclosure. The wound triaging and recommendation system 102 includes a hardware processor 216. The wound triaging and recommendation system 102 further includes a memory 202 coupled to the hardware processor 216. The memory 202 includes a set of program instructions in the form of a plurality of subsystems 108.

The hardware processor(s) 216, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 202 includes the plurality of subsystems 108 stored in the form of executable program which instructs the hardware processor 216 via a system bus 212 to perform the above-mentioned method steps. The plurality of subsystems 108 include following subsystems: a text and voice based conversational artificial intelligence (AI) subsystem 204, a wound image analytics subsystem 206, an AI based text and image analytics subsystem 208, and a patient treatment recommendation subsystem 210.

Computer memory elements may include any suitable memory device(s) for storing data and executable program, such as read only memory, random access memory, erasable programmable read only memory, electronically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 216.

The wound triaging and recommendation system 102 includes the text and voice based conversational artificial intelligence (AI) subsystem 204 that is communicatively connected to the hardware processor 216. The text and voice based conversational AI subsystem 204 obtains patient's medical data 302 including at least one of: history of one or more diseases of the patient 104, family information of the patient 104, symptoms of the one or more diseases in the patient 104, and medicines consumed by the patient 104 through the user device 106 of the patient 104. The text and voice based conversational artificial intelligence subsystem 204 determines data associated with at least one of: genetic predisposition, the one or more diseases influencing a wound, and a state of the wound based on an effect of a current medicine that the patient 104 consumes by assessing the obtained patient's medical data 302 using a machine learning algorithm including at least one of: random forests, logistic regression, support vector machines, neural networks, and the like.

The text and voice based conversational artificial intelligence (AI) subsystem 204 determines data associated with at least one of: the genetic predisposition, the one or more diseases influencing the wound, and the state of the wound by (a) storing the obtained patient's medical data 302, and (b) comparing the stored patient's medical data 302 with predetermined medical data to determine the data associated with at least one of: the genetic predisposition, the one or more diseases influencing the wound, and the state of the wound using the machine learning algorithm.

The wound triaging and recommendation system 102 further includes the wound image analytics subsystem 206 that is communicatively connected to the hardware processor 216. The wound image analytics subsystem 206 collects images of the wound 304 from the patient 104. The wound image analytics subsystem 206 analyzes the wound from the images of the wound 304 collected from the patient 104. In an embodiment, the patient's wound image 304 is captured using at least one of: a phone, a camera and any other image capturing means such as using specialized multi-spectral, hyperspectral in one or more wavelengths such as ultraviolet (UV), visible infrared (IR) and the like.

The wound image analytics subsystem 206 includes an artificial intelligence (AI) model based on machine and deep learning algorithm including at least one of: random forests, logistic regression, support vector machines, Bayesian algorithms, convolutional neural networks, generative adversarial networks, and the like, which analyses the patient's wound image 304. The wound image analytics subsystem 206 compares the collected images of the wound 304 with pre-classified images associated with the wound to classify the wound of the patient 104 into the plurality of categories using the machine learning algorithm. In an embodiment, the plurality of categories including at least one of: granulation, necrotic, and cellulitis are classified based on the collected images of the wound 304 from the patient 104 using the machine learning algorithm or the deep learning algorithm. The wound image analytics subsystem 206 finally determines severity and risk category of the wound based on the classification of the wound of the patient 104 using the machine learning algorithm or the deep learning algorithm.

The wound triaging and recommendation system 102 further includes the AI based text and image analytics subsystem 208 that is communicatively connected to the hardware processor 216. The AI based text and image analytics subsystem 208 obtains information associated with patient's clinical reports 306 from the patient 104. In an embodiment, the information associated with the patient's clinical reports 306 may include at least one of: an image, a text, and the like. The AI based text and image analytics subsystem 208 extracts key clinical parameters and changes in the key clinical parameters over time from the patient's clinical reports 306 by scanning the patient's clinical reports 306 using optical character recognition techniques.

The optical character recognition techniques for extracting the key clinical parameters and changes in the key clinical parameters over time from the patient's clinical reports 306 by (a) obtaining the information associated with the patient's clinical reports 306 from the patient 104 as at least one of: the image and the text, (b) pre-processing at least one of: the image and the text to extract non-zero pixels, (c) recognizing one or more characters based on the extracted non-zero pixels using segmentation, thresholding and AI based classification processes, and (d) post-processing the one or more characters to return at least one of: exact text and alphanumeric data including one or more numbers related to the key clinical parameters.

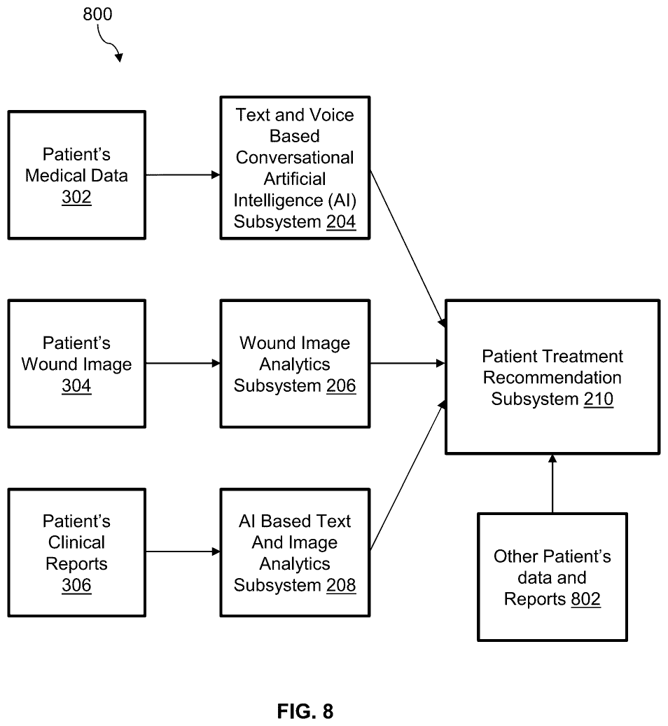

The wound triaging and recommendation system 102 further includes the patient treatment recommendation subsystem 210 that is communicatively connected to the hardware processor 216. The patient treatment recommendation subsystem 210 obtains at least one of: (a) the determined data associated with at least one of: the genetic predisposition, the one or more diseases influencing the wound, and the state of the wound based on the effect of the current medicine that the patient 104 consumes, (b) the determined severity and risk category of the wound, and (c) the extracted key clinical parameters and the changes in the key clinical parameters over time from at least one of: the text and voice based conversational AI subsystem 204, the wound image analytics subsystem 206, and the AI based text and image analytics subsystem 208. The patient treatment recommendation subsystem 210 further obtains medical data and reports 802 (shown in FIG. 8) from other patients. In an embodiment, the medical data and reports of other patients may include past medical history of the other patients.

The patient treatment recommendation subsystem 210 triages the wound to at least one of: identify the severity of the wound by qualifying risk score for the wound, provide wound prognostics for wound healing, and provide treatment recommendations associated with personalized therapeutic routes for healing the wound to the patient 104 based on results outputted from at least one of: the text and voice based conversational AI subsystem 204, the wound image analytics subsystem 206, the AI based text and image analytics subsystem 208, and the medical data and reports 802 from the other patients. In an embodiment, the personalized therapeutic routes provided by the patient treatment recommendation subsystem 210 may include at least one of: drugs, diet, lifestyle changes, antibiotics, topicals, wound dressing, negative wound pressure therapy, hyperbaric oxygen therapy, and wound debridement.

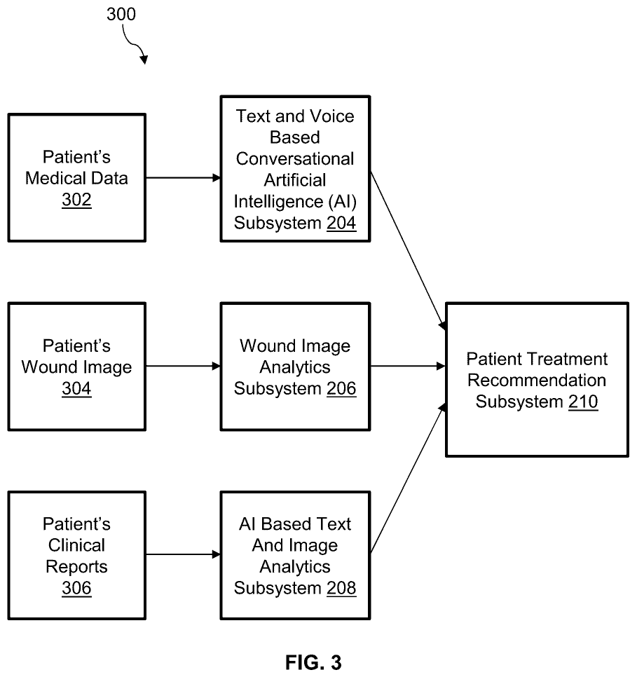

FIG. 3 is a block diagram 300 of the wound triaging and recommendation system 102, such as those shown in FIG. 1, in accordance with an embodiment of the present disclosure. The block diagram 300 of the wound triaging and recommendation system 102 includes the text and voice based conversational AI subsystem 204, the wound image analytics subsystem 206, the AI based text and image analytics subsystem 208, and the patient treatment recommendation subsystem 210. The functions of the above said subsystems are explained in FIG. 2.

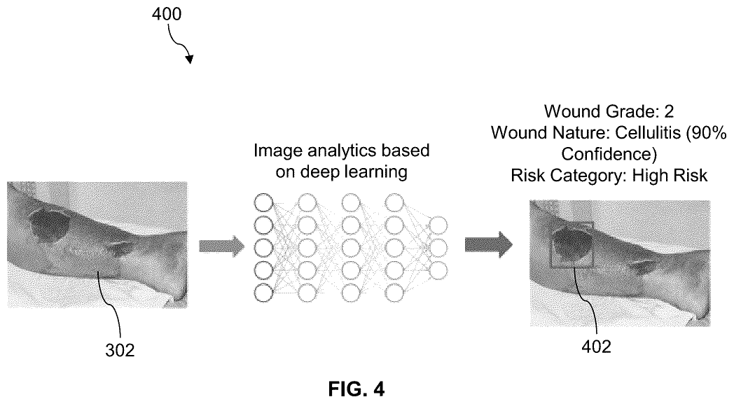

FIG. 4 is a schematic representation of an exemplary process flow depicting wound image analytics process 400, in accordance with an embodiment of the present disclosure. The wound image analytics subsystem 206 collects the patient's wound image 304. The patient's wound image 304 may be captured using at least one of: the phone, the other camera and any other image capturing means. The wound image analytics subsystem 206 includes an artificial intelligence (AI) system based on machine or deep learning algorithm (i.e., the machine or deep learning model). The artificial intelligence (AI) system based on machine or deep learning algorithm analyses the wound from the patient's wound image 304 classifies the wound into different categories (e.g., the granulation, the necrotic, the cellulitis, and the like) and grades severity and risk category 402 of the wound. For example, the grade 402 of the wound may be two, nature of the wound may be cellulitis with ninety percent confidence and the risk category may be of high risk.

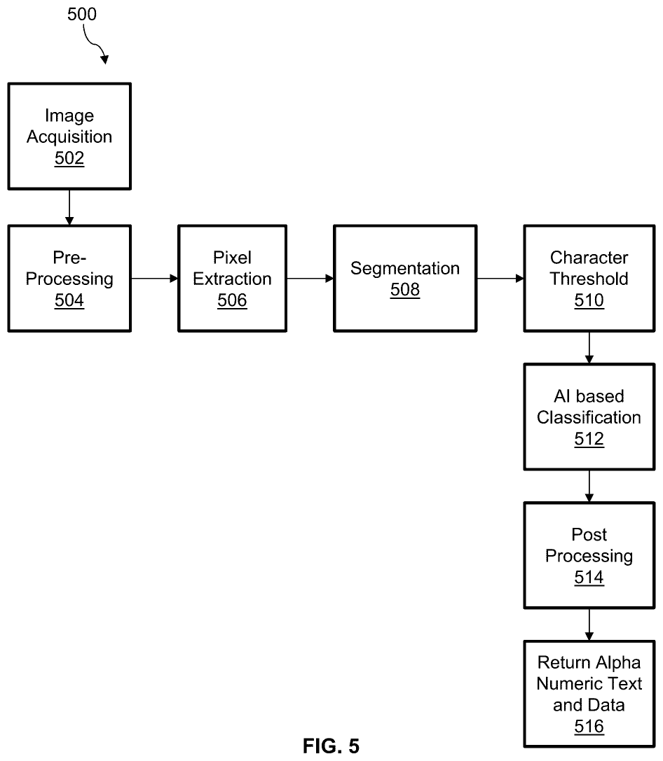

FIG. 5 is a process flowchart 500 illustrating an exemplary method of performing an artificial intelligence (AI) based image analytics, in accordance with an embodiment of the present disclosure. The artificial intelligence (AI) based text and image analytics subsystem 208 extracts the key clinical parameters from patients' clinical reports 306. The patients' clinical reports 306 are scanned using the optical character recognition techniques for extracting the key clinical parameters and the changes in the key clinical parameters over time. The optical character recognition technique includes of obtaining the information (i.e., image acquisition 502) associated with the patient's clinical reports 306 from the patient 104 as at least one of: an image and a text, image pre-processing (i.e., to improve the brightness, contrast, rotations etc.) 504 to extract 506 the non-zero pixels, followed by segmentation to recognize the regions of interest 508 and thresholding to removing the unwanted background 510, and an AI based classification system that uses machine and deep learning techniques 512 to recognize the characters. The characters are classified and post processed to enhanced recognition 514 finally to return 516 the exact text or alphanumeric data which also include of numbers related to various clinical parameters.

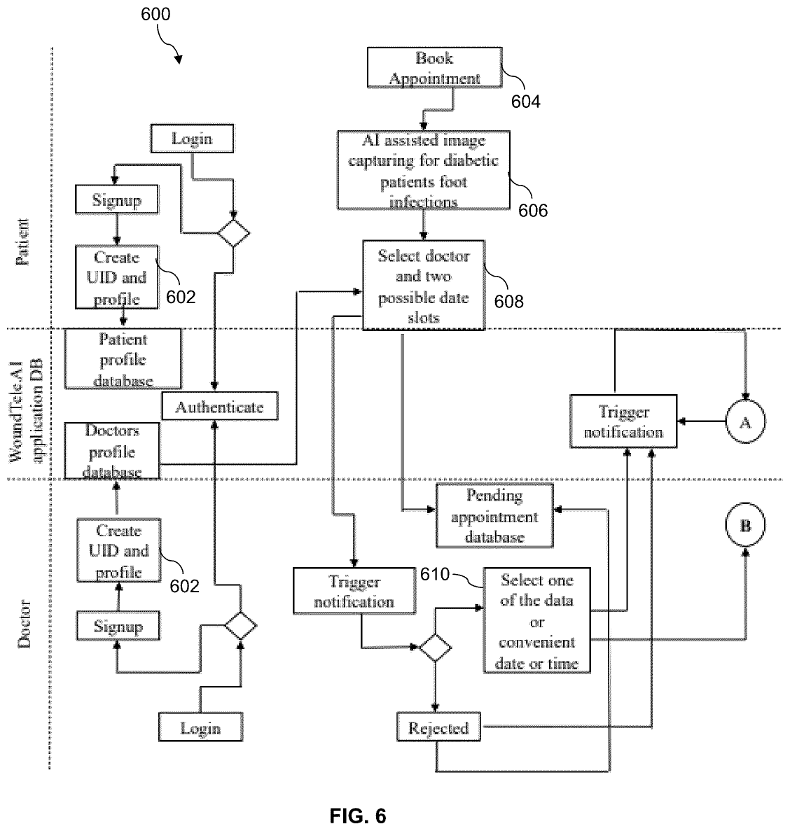

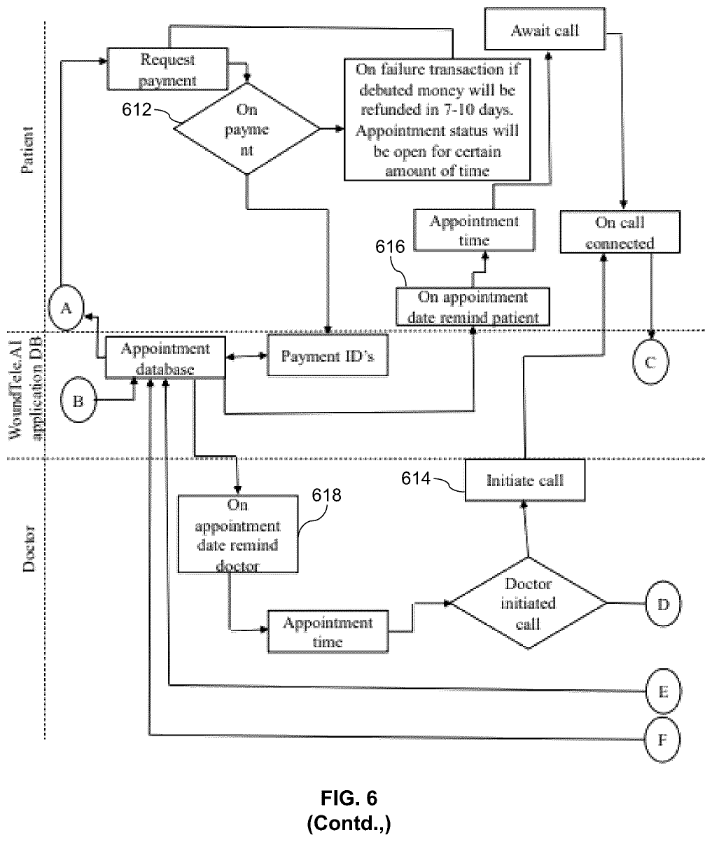

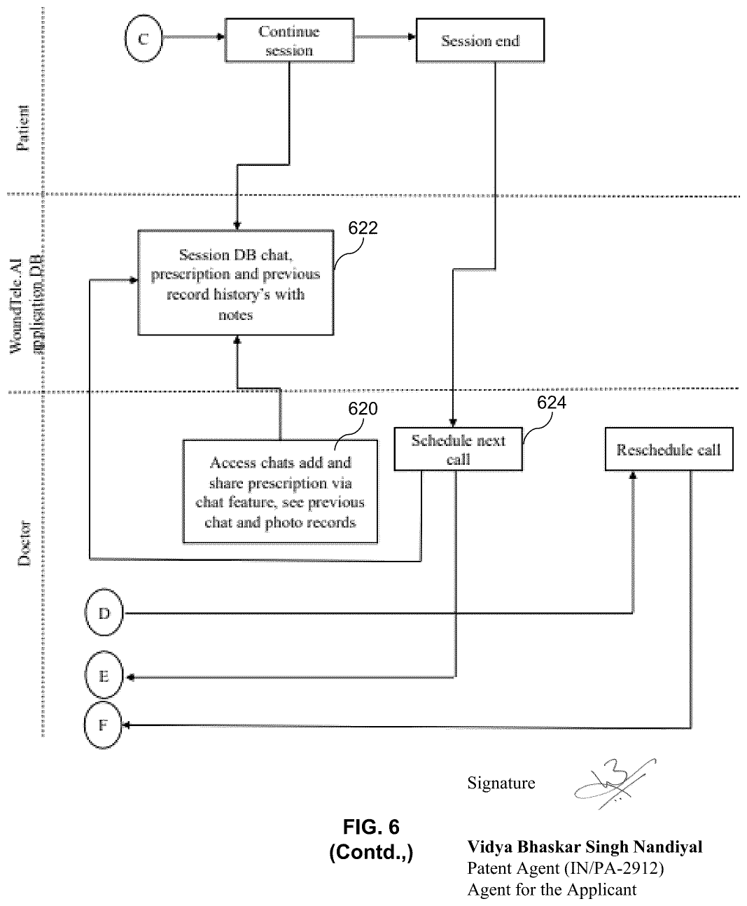

FIG. 6 is a process flowchart 600 illustrating an exemplary method of performing wound triaging and recommendation for treatments in case of digital assist or remote teleconsultation in a multi-user environment, in accordance with an embodiment of the present disclosure. The entire system 100 is implemented as a stand-alone virtual assist or as a digital assist to doctors for in-person visits or remote teleconsultation or video consultation of the patients 104. The system 100 comprises of connecting the patients 104 and the doctors for teleconsultation. Initially, the patient 104 is logged into the wound triaging and recommendation system 102. The wound triaging and recommendation system 102 may include an option to sign up by creating user ID (UID) and profiles if the patients 104 are new to the wound triaging and recommendation system 102. At step 602, the patients 104 can create a user ID, fill in the patient details and select the doctors for which teleconsultation is desired. The above-mentioned logging in and signing up process are same for the doctors. Subsequently, appointment with the doctors can be booked at step 604. Once the appointment is booked, the patient can take pictures of the wound image 304 and an artificial intelligence (AI) based system automatically triages the wound giving wound type, grade, and the like 402, and the like which can be directly viewed by the doctor, as shown in step 606. The patient 104 can select the doctors and at least two possible date slots for appointments with the doctors, as shown in step 608. The doctor also has a choice to confirm the appointment or to suggest alternative dates for the appointment, as shown in step 610.

A payment system is inbuilt for the patients to pay the teleconsultation fees in advance, as shown in step 612. The doctors can initiate the voice or video call on the agreed appointment date and can review the patient's history and artificial intelligence (AI) based recommendations, as shown in step 614. In addition, the doctors can also text or video chat with the patient and suggest prescriptions and other therapeutic recommendations taking the inputs from the digital artificial intelligence (AI) assist recommendation system (i.e., from the patient treatment recommendation subsystem 210). The artificial intelligence (AI) system also has explainability built in the wound triaging and recommendation system 102 so that the doctors understand the process by which the artificial intelligence (AI) system has arrived at the triaging and the recommendations.

In an embodiment, the wound triaging and recommendation system 102 sends reminder to the patients 104 when the appointment time is approached, as shown in step 616. In another embodiment, the wound triaging and recommendation system 102 sends reminder to the doctors when the appointment date is approached, as shown in step 618. The doctors can access the chats and share the prescription through a chat feature to the wound triaging and recommendation system 102 database, as shown in step 620. The wound triaging and recommendation system 102 database stores the session chat, the prescription, and previous medical record history with notes, as shown in step 622. In an embodiment, the doctors can schedule a next call with the patients 104, as shown in step 624. In another embodiment, the doctors can reschedule the call and the details of the rescheduled call with the patients 104 are stored in an appointment database.

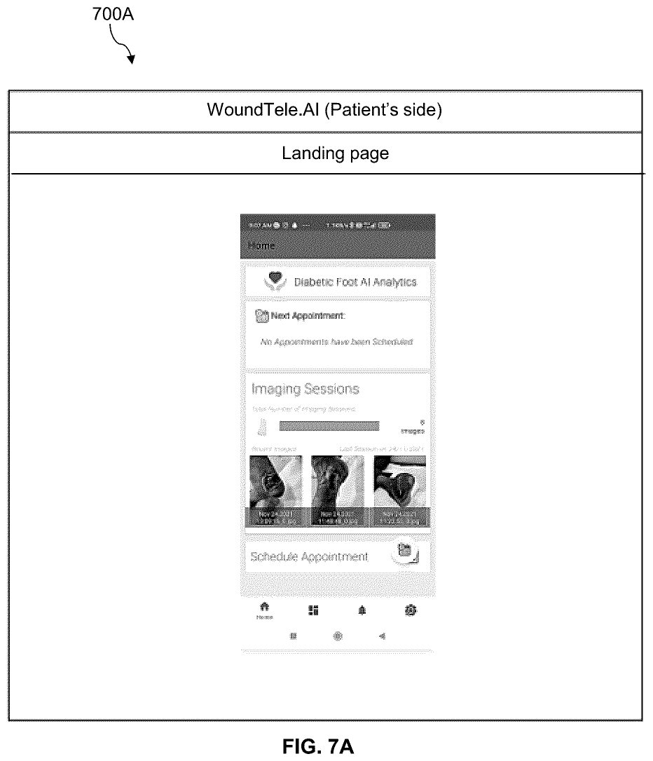

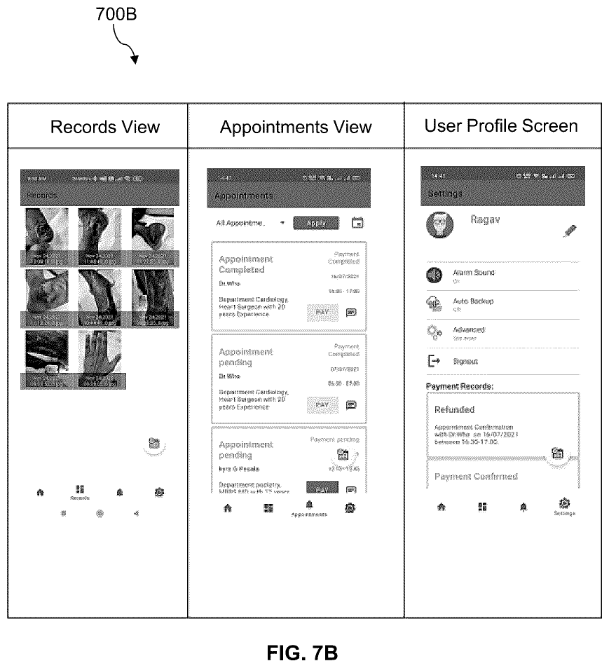

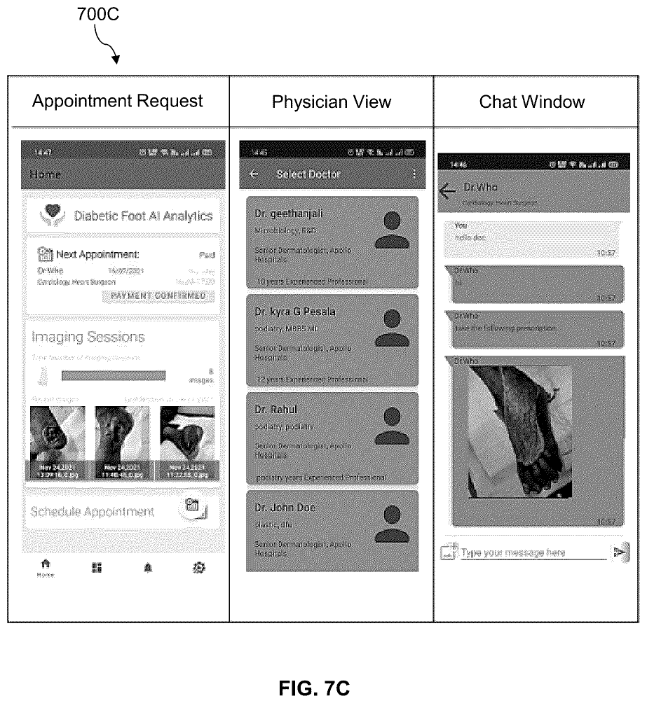

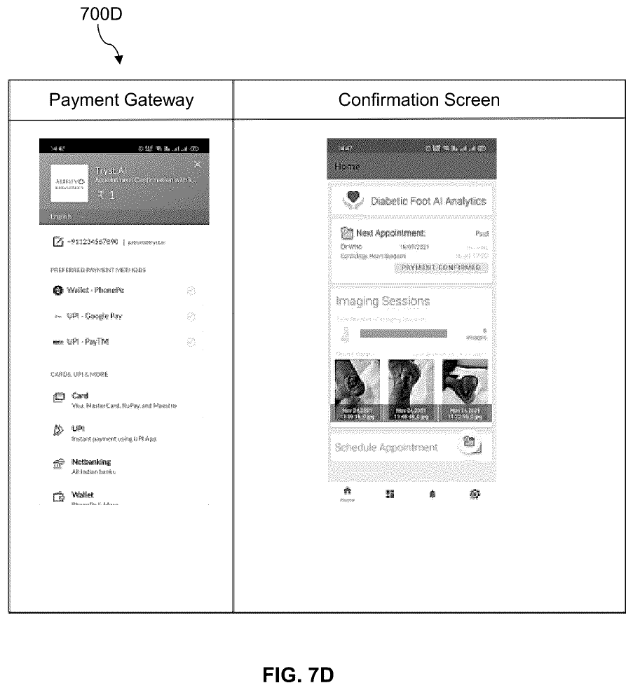

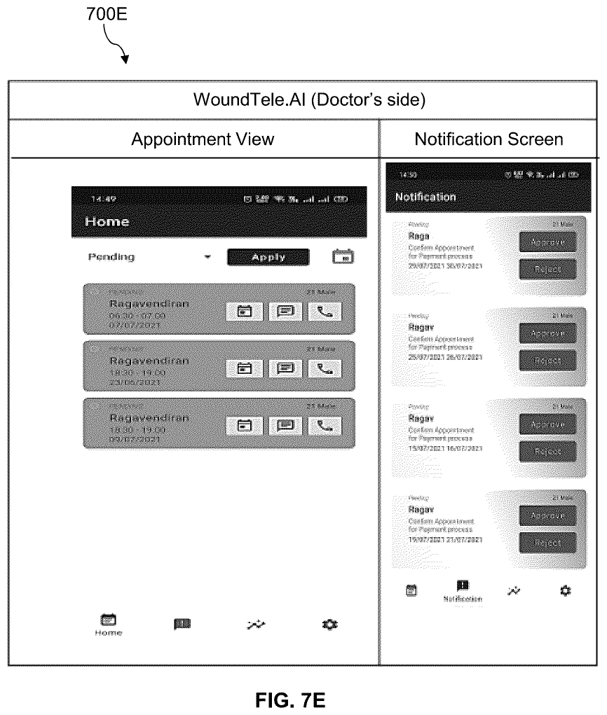

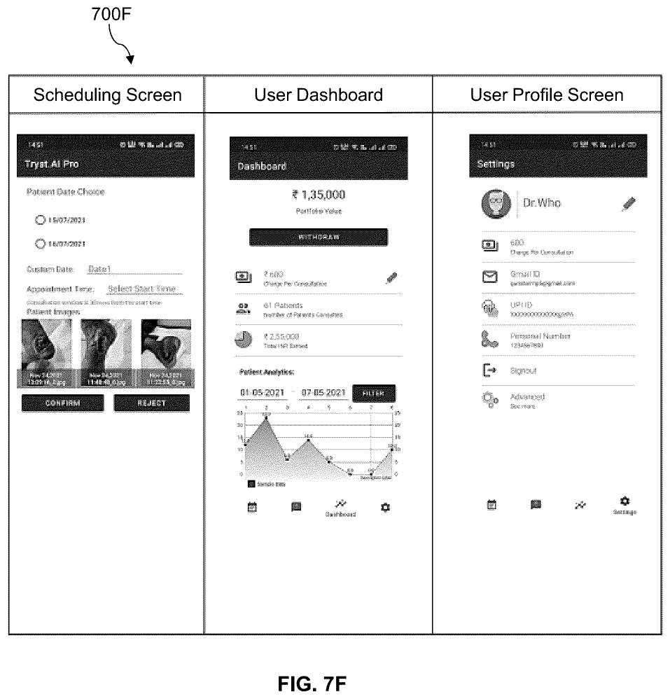

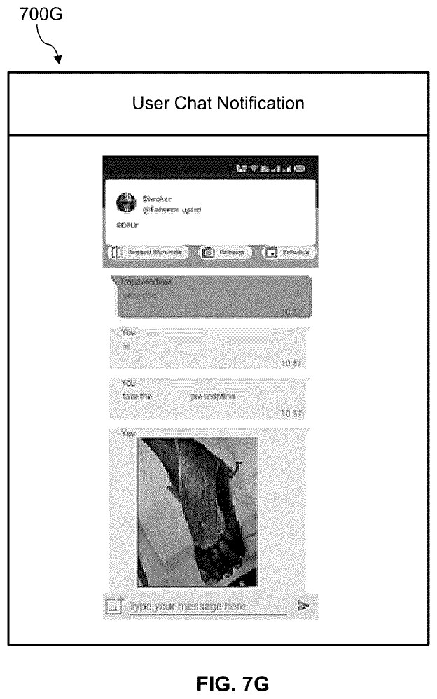

FIGS. 7A-7G are exemplary graphical user interfaces illustrating the wound triaging and recommendation system running on the user device 106, in accordance with an embodiment of the present disclosure. FIGS. 7A-7G depict an application running on the user device 106. For example, the application may be a software application installed on the user device 106 or may be web application hosted on a remote server (such as cloud or edge servers). The user device 106 may be at least one of: a phone, tablet, handheld device, computer, PC, desktop, and the like. FIGS. 7A-7D depict a dashboard of the application in case the user 104 is the patient 104. FIG. 7A is a graphical user interface view 700A that depicts a landing page of the application, which provides an appointment section, imaging sessions, and the like. FIG. 7B is a graphical user interface view 700B that depicts past records of the patient 104, appointments, and a user 104 (i.e., the patient 104) profile window. FIG. 7C is a graphical user interface view 700C that depicts appointment requests, physician view, and chat window. FIG. 7D is a graphical user interface view 700D that depicts a payment gateway, and a confirmation screen.

FIG. 7E is a graphical user interface view 700E that depicts a dashboard of the application in case the user 104 is the doctor. In this figure, an appointment view of the doctor and a notification screen is depicted. FIG. 7F is a graphical user interface view 700F that depicts a dashboard for a doctor including a scheduling screen, a user dashboard and a user profile screen. FIG. 7G is a graphical user interface view 700G that depicts a user chat and notification screen for the doctor.

FIG. 8 is a block diagram 800 of the patient treatment recommendation subsystem 210 based on the inputs collected from the patients' medical data 302, the wound image data 304, the clinical reports 306 and other patients medical data and reports 802, in accordance with an embodiment of the present disclosure. The patient treatment recommendation subsystem 210 receives inputs from the patients' medical data 302, the patient's wound image 304, the patients' clinical reports 306 and the other patients' medical data and reports 802. The inputs are received from the text and voice based conversational artificial intelligence (AI) subsystem 204, the wound imaging analytics subsystem 206, and the artificial intelligence (AI) based text and image analytics subsystem 208 respectively are combined for performing: firstly, wound triaging to qualify risk score, secondly to provide wound prognostics in terms of wound healing and finally to provide patient treatment recommendations from the patient treatment recommendation subsystem 210. The patient treatment recommendation subsystem 210 recommends personalised therapeutic routes such as for example, drugs, diet, lifestyle changes, antibiotics, topicals, wound dressing, and other modalities such as for example, negative wound pressure therapy, hyperbaric oxygen therapy, wound debridement, and the like. The recommendations to the patients 104 are also provided from the other patients' medical data and reports 802. The other patients' medical data and reports 802 may include the patient's past medical history. The explainable artificial intelligence (AI) framework is integrated with the system 100 for easy understanding of artificial intelligence (AI) decisions by doctors and the patients 104.

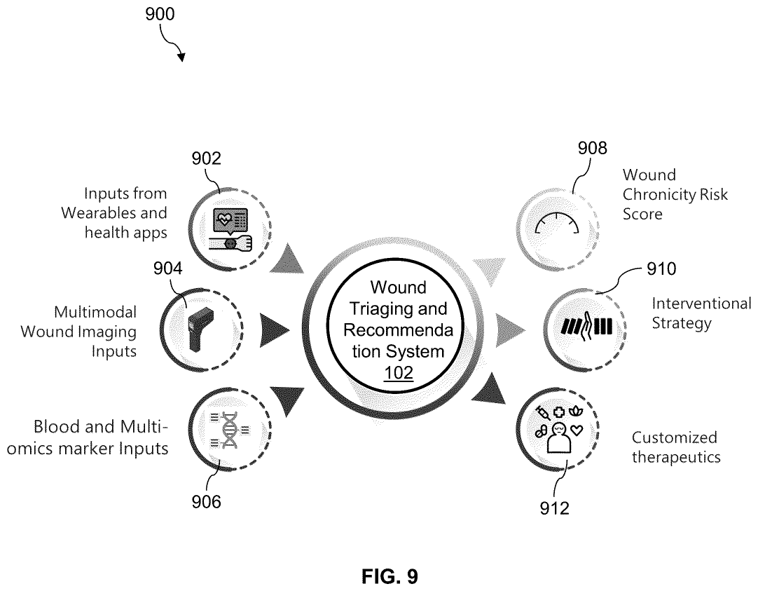

FIG. 9 is an exemplary view 900 of the wound triaging and recommendation system 102 (i.e., a wound digital assistant) to provide outputs of at least one of: a wound chronicity score 908, wound interventional strategies 910, and customized therapeutic strategies 912, in accordance with an embodiment of the present disclosure. The wound triaging and recommendation system 102 obtains inputs from at least one of: wearables and health applications 902, a multimodal wound imaging device 904, and blood and multi-omics markers 906. The wearables 900 may include at least one of: a smartwatch, a ring, and the like, for monitoring at least one of: glucose levels, vitals, and the like. Further, the health applications 902 may be related to at least one of: track vitals, body movement, and the like. The multimodal wound imaging device 904 may capture the images of the wound in at least one of white colour, fluorescence, near-infrared, thermal, and the like. The blood markers 906 may include at least one of: average blood glucose levels (Hba1c), inflammatory markers that include at least one of: Interleukin-6 (IL-6), c-reactive protein (CRP), and the like. The multi-omic markers 906 may include at least one of: genomic, epigenetic, metagenomic, transcriptomic, metabolomic, glycomic, and the like. The wound triaging and recommendation system 102 uses the machine learning algorithms including at least one of: random forest, k-nearest neighbour, the support vector machines, the deep learning algorithms such as the neural networks to provide the outputs of at least one of: the wound chronicity score 908, the wound interventional strategies 910, and the customized therapeutic strategies 912, based on the inputs. The wound interventional strategies 910 may include a debridement, a negative pressure wound therapy, a hyperbaric oxygenation therapy, and the like. In an embodiment, the customized therapeutic strategies 912 is used for effective wound management and healing that includes at least one of: wound dressings, topical ointments, antibiotics, and the like.

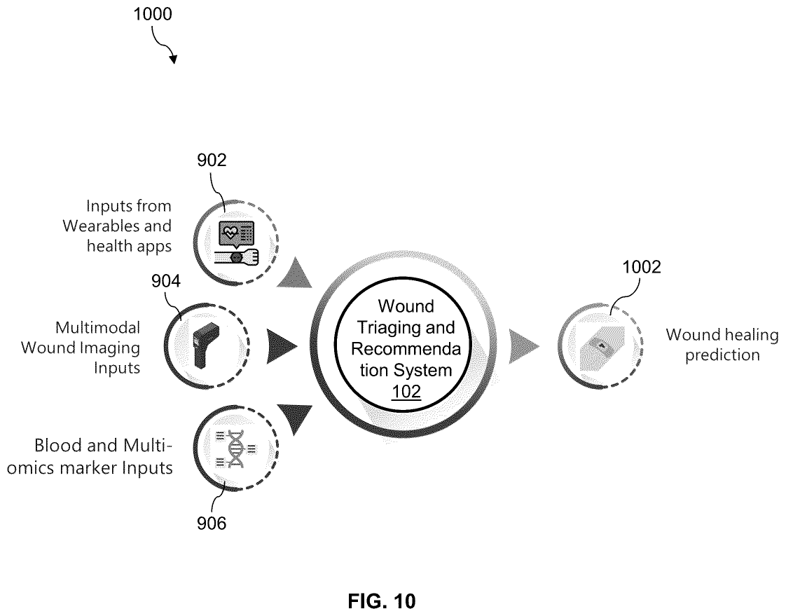

FIG. 10 is an exemplary view 1000 of the wound triaging and recommendation system 102 (i.e., the wound digital assistant) to predict wound healing probability and healing time 1002, in accordance with an embodiment of the present disclosure. The wound triaging and recommendation system 102 obtains inputs from at least one of: the wearables and the health applications 902, the multimodal wound imaging device 904, and the blood and multi-omics markers 906. The wearables 900 may include at least one of: the smartwatch, the ring, and the like, for monitoring at least one of: glucose levels, vitals, and the like. Further, the health applications 902 may be related to at least one of: the track vitals, the body movement, and the like. The multimodal wound imaging device 904 may capture the images of the wound in at least one of: the white colour, the fluorescence, the near-infrared, the thermal, and the like. The blood markers 906 may include at least one of: the average blood glucose levels (Hba1c), the inflammatory markers that include at least one of: the Interleukin-6 (IL-6), the c-reactive protein (CRP), and the like. The multi-omic markers 906 may include at least one of: the genomic, the epigenetic, the metagenomic, the transcriptomic, the metabolomic, the glycomic, and the like. The wound triaging and recommendation system 102 uses the machine learning algorithms including at least one of: the random forest, the k-nearest neighbour, the support vector machines, the Bayesian networks, the deep learning algorithms such as the neural networks to predict the wound healing probability and healing time 1002 based on the inputs. Further, the wound triaging and recommendation system 102 may predict the wound healing probability and healing time 1002 due to the wound interventional strategies that include at least one of: the debridement, the negative pressure wound therapy, the hyperbaric oxygenation therapy. Further, wound triaging and recommendation system 102 may predict the wound healing probability and healing time 1002 due to the therapeutic strategies that include at least one of: the wound dressings, the topical ointments, the antibiotics, and the like.

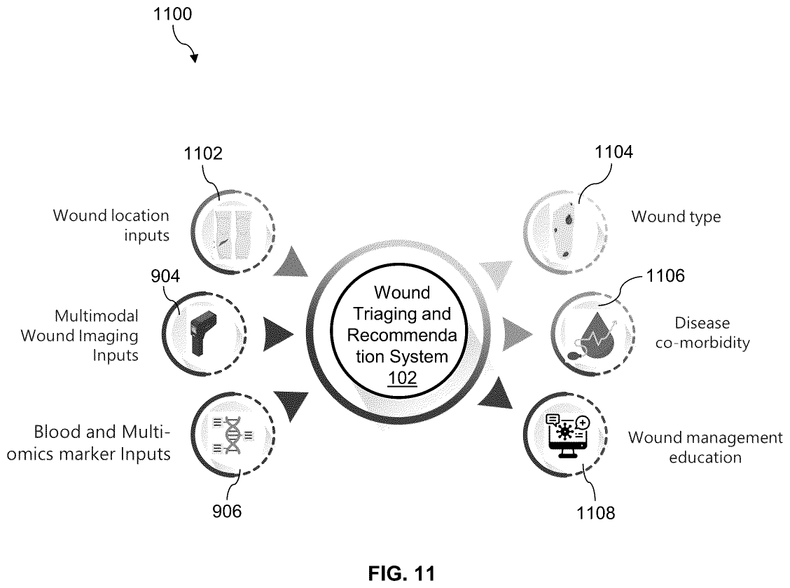

FIG. 11 is an exemplary view 1100 of the wound triaging and recommendation system 102 (i.e., the wound digital assistant) to predict a type of the wound 1104, and disease co-morbidity 1106, and to provide a wound management education 1108, in accordance with an embodiment of the present disclosure. The wound triaging and recommendation system 102 obtains inputs from at least one of: a location of the wound 1102, the multimodal wound imaging device 904, and the blood and multi-omics markers 906. The multimodal wound imaging device 904 may capture the images of the wound in at least one of: the white colour, the fluorescence, the near-infrared, the thermal, and the like. The blood markers 906 may include at least one of: the average blood glucose levels (Hba1c), the inflammatory markers that include at least one of: the Interleukin-6 (IL-6), the c-reactive protein (CRP), and the like. The multi-omic markers 906 may include at least one of: the genomic, the epigenetic, the metagenomic, the transcriptomic, the metabolomic, the glycomic, and the like. The wound triaging and recommendation system 102 uses the machine learning algorithms including at least one of: the random forest, the k-nearest neighbour, the support vector machines, the Bayesian networks, the deep learning algorithms such as the neural networks to predict the type of the wound 1104, and the disease co-morbidity 1106 based on the inputs. The type of the wound 1104 may include at least one of: diabetic foot ulcer, pressure ulcer, venous ulcer, and the like. The disease co-morbidity 1106 may include cardiac diseases including at least one of: atherosclerosis, hypertension, and the like. Further, the wound triaging and recommendation system 102 uses the machine learning algorithms to provide the wound management education 1108 to at least one of: the doctors, the physicians, and the patients 104 on best wound management strategies. The wound management strategies may include at least one of: the wound cleaning, the debridement, the dressings, the antibiotics, and the like. Further, the wound triaging and recommendation system 102 may connect with third-party labs and pathology service providers to enable geo-tagged customized services for effective wound management.

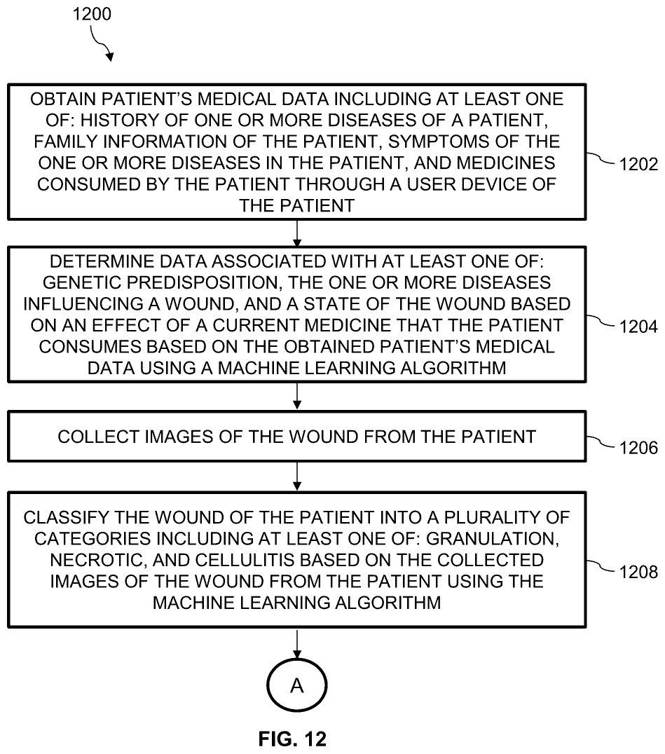

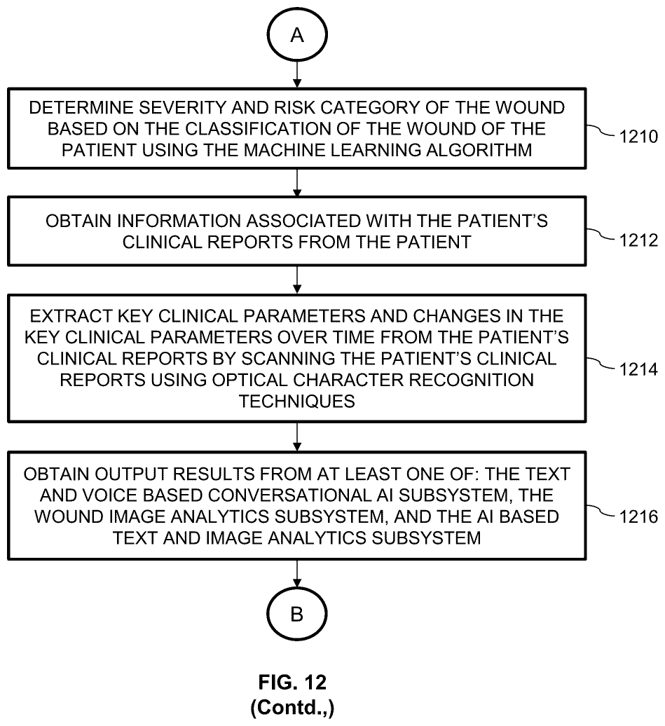

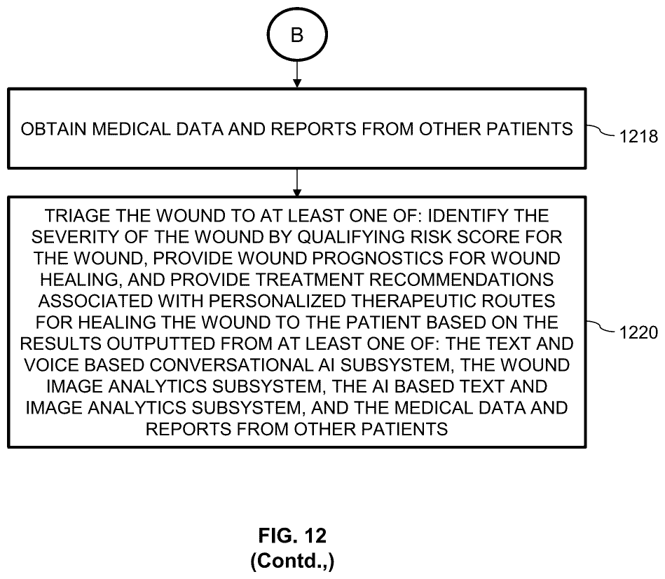

FIG. 12 is a flow chart illustrating a computer implemented wound triaging and recommendation method 1200 for wound triaging and recommendation for treatments using a wound triaging and recommendation system 102, in accordance with an embodiment of the present disclosure. At step 1202, the patient's medical data 302 including at least one of: history of one or more diseases of the patient 104, family information of the patient 104, symptoms of the one or more diseases in the patient 104, and medicines consumed by the patient 104 are obtained through the user device 106 of the patient 104. At step 1204, the data associated with at least one of: genetic predisposition, the one or more diseases influencing the wound, and the state of the wound based on the effect of the current medicine that the patient 104 consumes are determined based on the obtained patient's medical data 302 using the machine learning algorithm.

At step 1206, the images of the wound 304 are collected from the patient 104. At step 1208, the wound of the patient 104 is classified into the plurality of categories including at least one of: the granulation, the necrotic, and the cellulitis based on the collected images of the wound 304 from the patient 104 using the machine learning algorithm. At step 1210, severity and risk category of the wound are determined based on the classification of the wound of the patient 104 using the machine learning algorithm.

At step 1212, information associated with patient's clinical reports 306 are obtained from the patient 104. At step 1214, the key clinical parameters and the changes in the key clinical parameters over time are extracted from the patient's clinical reports 306 by scanning the patient's clinical reports 306 using optical character recognition techniques.

At step 1216, the at least one of: (a) the determined data associated with at least one of: the genetic predisposition, the one or more diseases influencing the wound, and the state of the wound based on the effect of the current medicine that the patient 104 consumes, (b) the determined severity and risk category of the wound, and (c) the extracted key clinical parameters and the changes in the key clinical parameters over time are obtained from the at least one of: the text and voice based conversational AI subsystem 204, the wound image analytics subsystem 206, and the AI based text and image analytics subsystem 208. At step 1218, the medical data and reports 802 are obtained from the other patients. In an embodiment, the medical data and reports 802 of the other patients may include the past medical history of the other patients.

At step 1220, the wound is triaged to at least one of: (a) identify the severity of the wound by qualifying risk score for the wound, (b) provide wound prognostics for wound healing, and (c) provide treatment recommendations associated with personalized therapeutic routes for healing the wound to the patient 104 based on results outputted from at least one of: the text and voice based conversational AI subsystem 204, the wound image analytics subsystem 206, the AI based text and image analytics subsystem 208, and the medical data and reports 802 from the other patients. In an embodiment, the personalized therapeutic routes provided by the patient treatment recommendation subsystem 210 may include at least one of: drugs, diet, lifestyle changes, antibiotics, topicals, wound dressing, negative wound pressure therapy, hyperbaric oxygen therapy, wound debridement, and the like.

In an embodiment, the present disclosure includes the following advantages. The present disclosure operates independently as a virtual doctor or may be presented as a digital assist to the doctor for in-person visits of the patients 104 or the digital assist for remote teleconsultation or video consultation of the patients 104. The present disclosure provides a virtual platform which enables the doctors and specialists to triage the wound from the patient's home.

The present disclosure further provides a solution to assist the doctors or the specialists to schedule appointments based on criticality of the wound of the patient 104 which is assessed by the present disclosure application's machine learning platform which classifies region of interest while imaging. The present disclosure provides a solution to track a patient's wound closure cycle by monitoring images of the wound 304 during the remote teleconsultation.

The present disclosure is also data driven and virtual framework for accurate diagnosis, prognosis, and therapeutics of the wounds. Additionally, digital assist for wound telemedicine enhances communication with a wound care specialist. The digital images captured are a safe, accurate and cost-effective referral pathway for skin lesions. The tele/video consulting may be used to be in touch with the patients 104 at home for continuous remote monitoring.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, and the like. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, and the like.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, and the like. of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. A wound triaging and recommendation system for wound triaging and recommendation for treatments, the wound triaging and recommendation system comprising:
a hardware processor; and
a memory coupled to the hardware processor, wherein the memory comprises a set of program instructions in the form of a plurality of subsystems, configured to be executed by the hardware processor, wherein the plurality of subsystems comprises:
a text and voice based conversational artificial intelligence (AI) subsystem configured to:
obtain patient's medical data comprising at least one of: history of one or more diseases of a patient, family information of the patient, symptoms of the one or more diseases in the patient, and medicines consumed by the patient (104) through a user device of the patient; and
determine data associated with at least one of: genetic predisposition, the one or more diseases influencing a wound, and a state of the wound based on an effect of a current medicine that the patient consumes based on the obtained patient's medical data using a machine learning algorithm;
a wound image analytics subsystem configured to:
collect images of the wound from the patient;
classify the wound of the patient into a plurality of categories comprising at least one of: granulation, necrotic, and cellulitis based on the collected images of the wound from the patient using the machine learning algorithm; and
determine severity and risk category of the wound based on the classification of the wound of the patient using the machine learning algorithm;
an AI based text and image analytics subsystem configured to:
obtain information associated with patient's clinical reports from the patient; and
extract key clinical parameters and changes in the key clinical parameters over time from the patient's clinical reports by scanning the patient's clinical reports using optical character recognition techniques; and
a patient treatment recommendation subsystem configured to:
obtain at least one of: (a) the determined data associated with at least one of: the genetic predisposition, the one or more diseases influencing the wound, and the state of the wound based on the effect of the current medicine that the patient consumes, (b) the determined severity and risk category of the wound, and (c) the extracted key clinical parameters and the changes in the key clinical parameters over time from at least one of: the text and voice based conversational AI subsystem, the wound image analytics subsystem, and the AI based text and image analytics subsystem;
obtain medical data and reports from other patients, wherein the medical data and reports of the other patients comprise past medical history of the other patients; and
triage the wound to at least one of: identify the severity of the wound by qualifying risk score for the wound, provide wound prognostics for wound healing, and provide treatment recommendations associated with personalized therapeutic routes for healing the wound to the patient based on results outputted from at least one of: the text and voice based conversational AI subsystem, the wound image analytics subsystem, the AI based text and image analytics subsystem, and the medical data and reports from other patients.

2. The wound triaging and recommendation system as claimed in claim 1, wherein in determining the data associated with at least one of: the genetic predisposition, the one or more diseases influencing the wound, and the state of the wound, the text and voice based conversational artificial intelligence (AI) subsystem is configured to:
store the obtained patient's medical data; and
compare the stored patient's medical data with predetermined medical data to determine the data associated with at least one of: the genetic predisposition, the one or more diseases influencing the wound, and the state of the wound using the machine learning algorithm.

3. The wound triaging and recommendation system as claimed in claim 1, wherein in classifying the wound of the patient, the wound image analytics subsystem is configured to:
analyze the wound from the images of the wound collected from the patient; and
compare the collected images of the wound with pre-classified images associated with the wound to classify the wound of the patient into the plurality of categories using the machine learning algorithm.

4. The wound triaging and recommendation system as claimed in claim 1, wherein in extracting the key clinical parameters and the changes in the key clinical parameters over time, the AI based text and image analytics subsystem is configured to:
obtain the information associated with the patient's clinical reports from the patient as at least one of: an image and a text;
pre-process at least one of: the image and the text to extract non-zero pixels;
recognize one or more characters based on the extracted non-zero pixels using segmentation, thresholding and AI based classification; and
post-process the one or more characters to return at least one of: exact text and alphanumeric data comprising one or more numbers related to the key clinical parameters.

5. The wound triaging and recommendation system as claimed in claim 1, wherein the personalized therapeutic routes provided by the patient treatment recommendation subsystem comprise at least one of: drugs, diet, lifestyle changes, antibiotics, topicals, wound dressing, negative wound pressure therapy, hyperbaric oxygen therapy, and wound debridement.

6. The wound triaging and recommendation system as claimed in claim 1, further comprising an explainable artificial intelligence (AI) framework, which enables physician, doctors and the patient to understand the wound triaging and the treatment recommendations outputted by the patient treatment recommendation subsystem.

7. The wound triaging and recommendation system as claimed in claim 1, wherein the images of the wound are captured using at least one of: a mobile phone, a camera, a specialized multi-spectral, and hyperspectral in one or more wavelengths comprising at least one of: ultraviolet (UV), and visible infrared (IR).

8. A wound triaging and recommendation method for wound triaging and recommendation for treatments using a wound triaging and recommendation system, the wound triaging and recommendation method comprising:
obtaining, by a hardware processor, patient's medical data comprising at least one of: history of one or more diseases of a patient, family information of the patient, symptoms of the one or more diseases in the patient, and medicines consumed by the patient through a user device of the patient;
determining, by the hardware processor, data associated with at least one of: genetic predisposition, the one or more diseases influencing a wound, and a state of the wound based on an effect of a current medicine that the patient consumes based on the obtained patient's medical data using a machine learning algorithm;
collecting, by the hardware processor, images of the wound from the patient;
classifying, by the hardware processor, the wound of the patient into a plurality of categories comprising at least one of: granulation, necrotic, and cellulitis based on the collected images of the wound from the patient using the machine learning algorithm;
determining, by the hardware processor, severity and risk category of the wound based on the classification of the wound of the patient using the machine learning algorithm;
obtaining, by the hardware processor, information associated with patient's clinical reports from the patient;
extracting, by the hardware processor, key clinical parameters and changes in the key clinical parameters over time from the patient's clinical reports by scanning the patient's clinical reports using optical character recognition techniques;
obtaining, by the hardware processor, at least one of: (a) determined data associated with at least one of: the genetic predisposition, the one or more diseases influencing the wound, and the state of the wound based on the effect of the current medicine that the patient consumes, (b) the determined severity and risk category of the wound, and (c) the extracted key clinical parameters and the changes in the key clinical parameters over time from at least one of: the text and voice based conversational AI subsystem, the wound image analytics subsystem, and the AI based text and image analytics subsystem;
obtaining, by the hardware processor, medical data and reports from other patients, wherein the medical data and reports of other patients comprise past medical history of the other patients; and
triaging, by the hardware processor, the wound to at least one of: identify the severity of the wound by qualifying risk score for the wound, provide wound prognostics for wound healing, and provide treatment recommendations associated with personalized therapeutic routes for healing the wound to the patient based on results outputted from at least one of: the text and voice based conversational AI subsystem, the wound image analytics subsystem, the AI based text and image analytics subsystem, and the medical data and reports from other patients.

9. The wound triaging and recommendation method as claimed in claim 8, wherein determining the data associated with at least one of: the genetic predisposition, the one or more diseases influencing the wound, and the state of the wound comprises:
storing, by the hardware processor, the obtained patient's medical data; and
comparing, by the hardware processor, the stored patient's medical data with predetermined medical data to determine the data associated with at least one of: the genetic predisposition, the one or more diseases influencing the wound, and the state of the wound using the machine learning algorithm.

10. The wound triaging and recommendation method as claimed in claim 8, wherein classifying the wound of the patient comprises:
analyzing, by the hardware processor, the wound from the images of the wound collected from the patient; and
comparing, by the hardware processor, the collected images of the wound with pre-classified images associated with the wound to classify the wound of the patient into the plurality of categories using the machine learning algorithm.

11. The wound triaging and recommendation method as claimed in claim 8, wherein extracting the key clinical parameters and the changes in the key clinical parameters over time comprises:
obtaining, by the hardware processor, the information associated with the patient's clinical reports from the patient as at least one of: an image and a text;
pre-processing, by the hardware processor, at least one of: the image and the text to extract non-zero pixels;
recognizing, by the hardware processor, one or more characters based on the extracted non-zero pixels using segmentation, thresholding and AI based classification; and
post-processing, by the hardware processor, the one or more characters to return at least one of: exact text and alphanumeric data comprising one or more numbers related to the key clinical parameters.

12. The wound triaging and recommendation method as claimed in claim 8, wherein the personalized therapeutic routes provided by the patient treatment recommendation subsystem comprise at least one of: drugs, diet, lifestyle changes, antibiotics, topicals, wound dressing, negative wound pressure therapy, hyperbaric oxygen therapy, and wound debridement.
